(19) United States
(12) Patent Application Publication
(10) Pub. No.: US 2017/0004626 A1
(43) Pub. Date: Jan. 5, 2017

(54) IMAGE PROCESSING DEVICE, IMAGE PROCESSING METHOD, AND COMPUTER-READABLE RECORDING MEDIUM

(71) Applicant: OLYMPUS CORPORATION, Tokyo (JP)

(72) Inventor: Satomi KOBAYASHI, Tokyo (JP)

(73) Assignee: OLYMPUS CORPORATION, Tokyo (JP)

(21) Appl. No.: 15/268,547

(22) Filed: Sep. 17, 2016

Related U.S. Application Data
(63) Continuation of application No. PCT/JP2015/062844, filed on Apr. 28, 2015.

(30) Foreign Application Priority Data
Sep. 29, 2014 (JP) 2014-199084

(51) Int. Cl. G06T 7/00; G06T 7/40; G06T 7/60; A61B 1/04

(52) U.S. Cl. CPC G06T 7/0081; A61B 1/041; G06T 7/408; G06T 7/60; G06T 7/0097; G06T 2207/10068; G06T 2207/20182; G06T 2207/20224; G06T 2207/30004

(57) ABSTRACT

An image processing device includes a processor including hardware. The processor is configured to: detect an area not suitable for an observation inside each image included in an image group in which images are acquired in time sequence; calculate an importance of the image for each of the images included in the image group based on the area not suitable for the observation inside the image; integrate the importance in order of time sequence; and determine whether the integrated value exceeds a threshold value.

### CROSS REFERENCES TO RELATED APPLICATIONS

This application is a continuation of PCT international application Ser. No. PCT/JP2015/062844, filed on Apr. 28, 2015 which designates the United States, incorporated herein by reference, and which claims the benefit of priority from Japanese Patent Applications No. 2014-199084, filed on Sep. 29, 2014, incorporated herein by reference.

### BACKGROUND

### 1. Technical Field

The disclosure relates to an image processing device, an image processing method, and a computer-readable recording medium.

### 2. Related Art

Hitherto, there is known an image extraction device (an image processing device) which acquires an image group including a plurality of images acquired in time sequence and performs an image summarization process of extracting a part of images from the image group so as to summarize in an image group including fewer images than the images included in the original image group (for example, see JP 2009-5020 A).

In the image processing device disclosed in JP 2009-5020 A, an image at a position in which a scene changes is selected as a representative image from the image group and the image group is summarized in a predetermined number of representative images.

Then, a user can recognize the content of the entire original image group in a short time by observing a predetermined number of representative images included in the image group subjected to the image summarization process.

### SUMMARY

In some embodiments, an image processing device includes a processor including hardware. The processor is configured to: detect an area not suitable for an observation inside each image included in an image group in which images are acquired in time sequence; calculate an importance of the image for each of images included in the image group based on the area not suitable for the observation inside the image; integrate the importance in order of time sequence; and determine whether the integrated value exceeds a threshold value.

In some embodiments, an image processing method executed by an image processing device includes: detecting an area not suitable for an observation inside an image included in an image group in which images are acquired in time sequence; calculating an importance of the image included in the image group based on the area not suitable for the observation inside the image; integrating the importance in order of time sequence; and determining whether the integrated value exceeds a threshold value.

In some embodiments, a non-transitory computer-readable recording medium recording a program for causing an image processing device to execute the image processing method is provided.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic diagram illustrating an endoscope system according to a first embodiment of the invention;

FIG. 2 is a block diagram illustrating an image processing device illustrated in FIG. 1;

FIG. 3 is a flowchart illustrating an operation (an image processing method) of the image processing device illustrated in FIG. 2;

FIG. 4 is a diagram illustrating an example of a representative image selected by the image processing method illustrated in FIG. 3;

FIG. 5 is a diagram illustrating an example of a representative image selected by the image processing method illustrated in FIG. 3;

FIG. 6 is a flowchart illustrating an image processing method according to a second embodiment of the invention;

FIG. 7 is a diagram illustrating an example of a representative image selected by the image processing method illustrated in FIG. 6; and

FIG. 8 is a diagram illustrating an example of a representative image selected by the image processing method illustrated in FIG. 6.

### DETAILED DESCRIPTION

Hereinafter, preferred embodiments of an image processing device, an image processing method, and an image processing program according to the invention will be described in detail with reference to the drawings. In addition, the invention is not limited by the embodiments.

### First Embodiment

### Schematic Configuration of Endoscope System

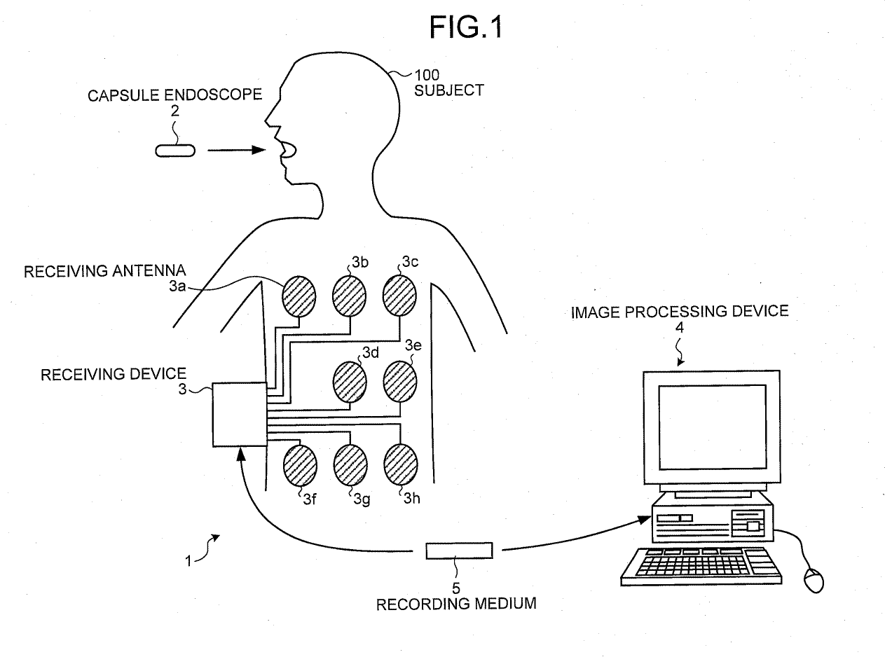

FIG. 1 is a schematic diagram illustrating an endoscope system 1 according to a first embodiment of the invention.

The endoscope system 1 is a system that acquires an in-vivo image inside a subject 100 by using a swallow type capsule endoscope 2 and allows a doctor or the like to observe the in-vivo image.

As illustrated in FIG. 1, the endoscope system 1 includes a receiving device 3, an image processing device 4, and a portable recording medium 5 other than the capsule endoscope 2.

The recording medium 5 is a portable recording medium that delivers data between the receiving device 3 and the image processing device 4 and is attachable to or detachable from each of the receiving device 3 and the image processing device 4.

The capsule endoscope 2 is a capsule endoscope device which is formed in a size in which the capsule endoscope can be introduced into an organ of the subject 100 and sequentially captures the in-vivo image while being introduced into the organ of the subject 100 by oral intake or the like and moved inside the organ by peristaltic motion or the like. Then, the capsule endoscope 2 sequentially transmits image data generated by the capturing operation.

The receiving device 3 includes a plurality of receiving antennas 3a to 3h and receives the image data from the capsule endoscope 2 inside the subject 100 through at least one of the plurality of receiving antennas 3a to 3h. Then, the receiving device 3 stores the received image data inside the recording medium 5 inserted into the receiving device 3.

Further, as illustrated in FIG. 1, the receiving antennas 3a to 3h may be disposed on a body of the subject 100 or may be disposed in a jacket to be worn by the subject 100. Further, the number of the receiving antennas of the receiving device 3 is not particularly limited to eight as long as the number is one or more.

### Configuration of Image Processing Device

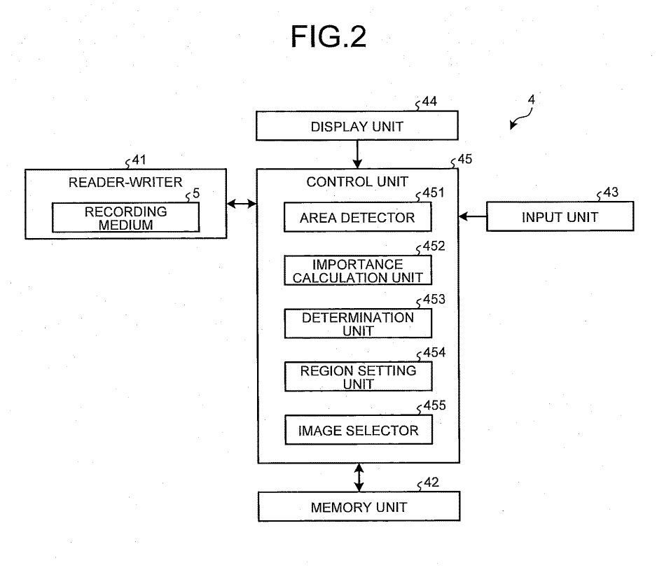

FIG. 2 is a block diagram illustrating the image processing device 4.

The image processing device 4 is configured as a workstation that acquires the image data inside the subject 100 and displays an image corresponding to the acquired image data.

As illustrated in FIG. 2, the image processing device 4 includes a reader-writer 41, a memory unit 42, an input unit 43, a display unit 44, and a control unit 45.

The reader-writer 41 serves as an image acquiring unit that acquires image data as a processing object from the outside.

Specifically, when the recording medium 5 is inserted into the reader-writer 41, the reader-writer 41 receives image data (an in-vivo image group including a plurality of in-vivo images captured (acquired) in time sequence by the capsule endoscope 2) stored in the recording medium 5 under the control of the control unit 45. Further, the reader-writer 41 transfers the received in-vivo image group to the control unit 45. Then, the in-vivo image group transferred to the control unit 45 is stored in the memory unit 42.

The memory unit 42 stores the in-vivo image group transferred from the control unit 45. Further, the memory unit 42 stores various programs (including an image processing program) executed by the control unit 45 or information necessary for the process of the control unit 45.

The input unit 43 is configured as a keyboard and a mouse and receives an operation of a user.

The display unit 44 is configured as a liquid crystal display or the like and displays a display screen (for example, a display screen including a predetermined number of representative images selected by an image summarization process to be described later) including the in-vivo image under the control of the control unit 45.

The control unit 45 is configured as a CPU (Central Processing Unit) or the like and reads a program (including an image processing program) stored in the memory unit 42 so as to control the operation of the entire image processing device 4 according to the program.

Hereinafter, a function that performs the “image summarization process” as a main part of the disclosure will be mainly described as the function of the control unit 45.

The control unit 45 includes, as illustrated in FIG. 2, an area detector 451, an importance calculation unit 452, a determination unit 453, a region setting unit 454, and an image selector 455.

The area detector 451 detects an invalid area (an area not suitable for an observation) other than a valid area suitable for an observation inside the in-vivo image by the unit of pixel every in-vivo image included in the in-vivo image group stored in the memory unit 42.

Specifically, the area detector 451 compares a feature data representing color information, frequency information, shape information, and the like which can be acquired from the in-vivo image with a second threshold value and detects the invalid area other than the valid area suitable for the observation inside the in-vivo image based on the comparison result.

Here, the valid area indicates an area in which a mucosa on a living body, a blood vessel, and a blood are reflected. Meanwhile, the invalid area is an area other than the valid area and indicates an area in which residue or bubbles are reflected, an area (a dark part) in which a deep part of a lumen is reflected, a halation area (a bright part) which is reflected as a mirror surface from a surface of an object, an area in which noise is generated by a communication error between the capsule endoscope 2 and the receiving device 3, and the like.

In addition, as the above-described invalid area detection method, various known methods can be employed (for example, JP 2007-313119 A, JP 2011-234931 A, JP 2010-115413 A, and JP 2012-16454 A).

The importance calculation unit 452 calculates the importance of the in-vivo image based on the invalid area detected by the area detector 451 inside the in-vivo image every in-vivo image included in the in-vivo image group stored in the memory unit 42.

Specifically, the importance calculation unit 452 calculates the importance of the in-vivo image based on the area (the number of pixels detected as the invalid area by the area detector 451) of the invalid area inside the in-vivo image. Here, the importance calculation unit 452 sets the importance of the in-vivo image to a high value as the number of pixels detected as the invalid area by the area detector 451 decreases.

The determination unit 453 calculates the importance of each in-vivo image calculated by the importance calculation unit 452 in time sequence and determines whether the calculated value exceeds a first threshold value (corresponding to a threshold value of the invention).

Specifically, the determination unit 453 divides the sum of the importance of all in-vivo images included in the in-vivo image group by the number of the representative images to be selected and sets the divided value as the first threshold value. Then, the determination unit 453 integrates the importance of all in-vivo images in order of time sequence and compares the integrated value with the first threshold value so as to determine whether the integrated value exceeds the first threshold value.

Further, the time-sequence calculation of the importance is not limited to the integration and the other calculation method may be also used. Further, the first threshold value may be also calculated by an arithmetic expression in response to the importance calculation method.

The region setting unit 454 sets a plurality of selection ranges respectively dividing all in-vivo images included in the in-vivo image group including the in-vivo images continuous in order of time sequence into a plurality of groups based on the determination result of the determination unit 453.

Specifically, the region setting unit 454 sets the frame number of the in-vivo image obtained when the determination unit 453 determines that the integrated value exceeds the first threshold value as the boundary of the selection range.

The image selector 455 selects the representative image from each of the in-vivo images included in the selection range in each of the selection ranges.

Further, in the first embodiment, the image selector 455 selects the in-vivo image served as the boundary of the selection range as the representative image among the in-vivo images included in the selection range.

### Operation of Image Processing Device

Next, the operation (the image processing method) of the above-described image processing device 4 will be described.

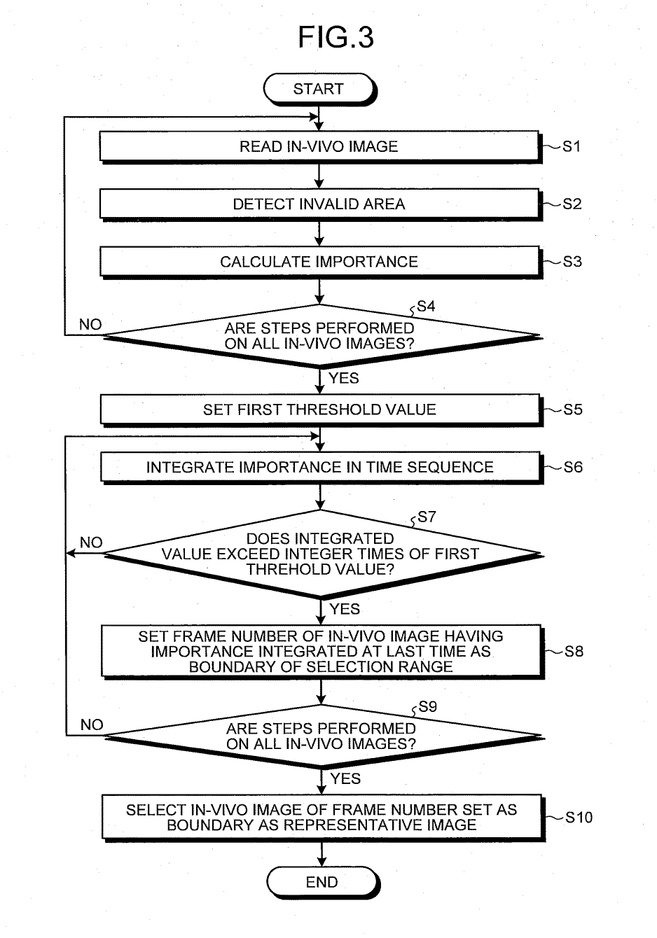

FIG. 3 is a flowchart illustrating the operation (the image processing method) of the image processing device 4.

In the description below, it is assumed that the recording medium 5 is inserted into the reader-writer 41, the in-vivo image group stored in the recording medium 5 is received via the reader-writer 41, and the in-vivo image group is stored in the memory unit 42 in advance.

First, the control unit 45 reads all in-vivo images included in the in-vivo image group stored in the memory unit 42 in order of time sequence (the frame number order) one by one (step S1).

Next, the area detector 451 detects the invalid area inside the in-vivo image read in step S1 every in-vivo image (step S2: an area detection step).

Next, the importance calculation unit 452 calculates the importance of the in-vivo image Pᵢ (i=frame number) of the in-vivo image by the following Equation (1) based on the invalid area inside the in-vivo image detected in step S2 every in-vivo image (step S3: an importance calculation step). Then, the importance calculation unit 452 stores the calculated importance Pᵢ in the memory unit 42 so that the calculated importance is correlated with the in-vivo image of the corresponding frame number.

Further, in Equation (1), the count (All in-vivo images) indicates the number of all pixels of the in-vivo image. The count (Invalid area) indicates the area (the number of pixels detected as the invalid area) of the invalid area detected by the area detector 451.

pᵢ=1−count(Invalid area)/count(All in-vivo images) (1)

As understood from Equation (1), the importance calculation unit 452 calculates a ratio of the number of pixels set as the valid area with respect to the number of all pixels of the in-vivo image as the importance Pᵢ. For example, when all in-vivo images are in the invalid area, the importance Pᵢ is set as “0”. Further, when all in-vivo images are in the valid area (when there is no invalid area), the importance Pᵢ is set as “1”. That is, the importance calculation unit 452 sets the importance of the in-vivo image Pᵢ to a high value as the number of pixels detected as the invalid area in the area detector 451 decreases.

Next, the control unit 45 determines whether step S1 to step S3 are performed on all in-vivo images included in the in-vivo image group stored in the memory unit 42 (step S4).

When it is determined that the above-described steps are not performed on all in-vivo images (step S4: No), the control unit 45 returns a routine to step S1 and sequentially calculates the importance of the other in-vivo images.

Meanwhile, when it is determined that the above-described steps are performed on all in-vivo images (step S4: Yes), the determination unit 453 divides the sum of the importance Pᵢ of all in-vivo images included in the in-vivo image group stored in the memory unit 42 by the number of the representative images to be calculated and sets the divided value as the first threshold value (step S5). Then, the determination unit 453 stores the first threshold value in the memory unit 42.

Next, the determination unit 453 integrates the importance Pᵢ of all in-vivo images included in the in-vivo image group stored in the memory unit 42 in order of time sequence (step S6) and determines whether the integrated value exceeds the integral multiple of the first threshold value (step S7).

Step S6 and step S7 described above correspond to a determination step according to the invention.

When it is determined that the integrated value does not exceed the integral multiple of the first threshold value (step S7: No), the determination unit 453 returns a routine to step S6 and continuously integrates the importance Pᵢ.

Meanwhile, when it is determined that the integrated value exceeds the integral multiple of the first threshold value (step S7: Yes), the region setting unit 454 sets the frame number of the in-vivo image having the importance Pᵢ integrated at the last time when the integrated value exceeds the integral multiple of the first threshold value as the boundary of the selection range (step S8). Then, the region setting unit 454 stores the frame number set as the boundary in the memory unit 42.

Next, the region setting unit 454 determines whether step S6 to step S8 are performed on all in-vivo images included in the in-vivo image group stored in the memory unit 42 (whether the importance are integrated to the importance Pᵢ of the in-vivo image having the largest frame number included in the in-vivo image group) (step S9).

When it is determined that the above-described steps are not performed on all in-vivo images (step S9: No), a routine returns to step S6 and the determination unit 453 continuously integrates the importance Pᵢ.

Meanwhile, when it is determined that the above-described steps are performed on all in-vivo images (step S9: Yes), the image selector 455 reads the frame number (the frame number set as the boundary in step S8) stored in the memory unit 42 and selects the in-vivo image of the frame number as the representative image (step S10).

### Detailed Example of Selected Representative Image

Next, a detailed example of the representative image selected by the above-described image processing method will be described.

Hereinafter, for the convenience of description, a range (a zone) in which the in-vivo image (the in-vivo image of which the importance Pᵢ is equal to or larger than “0.6”) having a small number of invalid areas is arranged and a range (a zone) in which the in-vivo image (the in-vivo image of which the importance Pᵢ is smaller than “0.6”) having a large number of invalid areas is arranged will be described in order.

### Arrangement Range of In-vivo Image Having Small Invalid Area

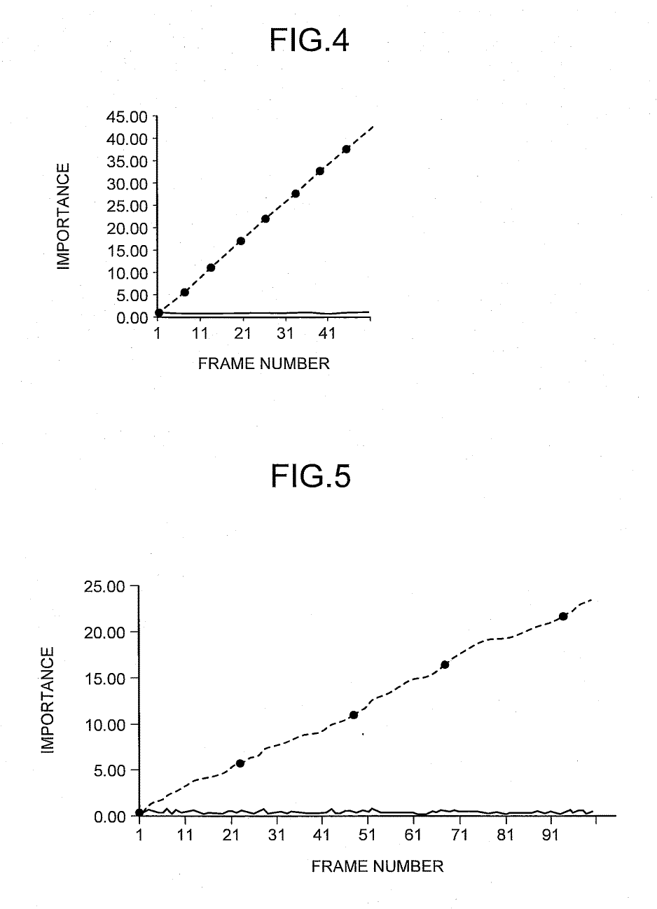

FIG. 4 is a diagram illustrating an example of the representative image selected by the above-described image processing method. Specifically, FIG. 4 illustrates an exemplary case in which two thousand representative images are selected from the in-vivo image group including sixty thousand in-vivo images. Here, the importance Pᵢ (depicted as a solid line in FIG. 4) and the integrated value (depicted as a dashed line in FIG. 4) are written in each frame number of the in-vivo images included in the in-vivo image group. Further, in FIG. 4, only the range of the frame numbers “1” to “50” of the arranged in-vivo images having a small number of invalid areas is depicted and the other ranges are omitted. Further, in FIG. 4, the selected representative image is expressed as a black circle.

In the example illustrated in FIG. 4, the sum of the importance Pᵢ of sixty thousand in-vivo images is “10600.00”. Then, the number of the representative images to be selected is “2000”. For this reason, in step S5, “5.3” as a value obtained by dividing “10600.00” by “2000” is set as a first threshold value T.

Even when the importance of the frame number “1” is any value, the importance exceeds “0” as zero times of the first threshold value T. For this reason, in step S8, the frame number “1” is set as the boundary of the selection range. That is, in step S10, the in-vivo image of the frame number “1” is selected as the representative image.

The integrated value (the integrated value of the importance Pᵢ of the frame numbers “1” to “7”) at the time point of the frame number “7” is “5.57” and exceeds “5.3” as one time of the first threshold value T. For this reason, in step S8, the frame number “7” is set as the boundary of the selection range. That is, in step S10, the in-vivo image of the frame number “7” is selected as the representative image.

Similarly, in step S10, the in-vivo images of the frame number “13”, “19”, “26”, “32”, “38”, “45”, and the like set as the boundary of the selection range are selected as the representative images.

### Arrangement Range of In-Vivo Image Having Large Invalid Area

FIG. 5 is a diagram illustrating an example of the representative image selected by the above-described image processing method. Specifically, FIG. 5 illustrates an exemplary case in which two thousand representative images are selected from the in-vivo image group (the in-vivo image group different from the in-vivo image group illustrated in FIG. 4) including sixty thousand in-vivo images similarly to the example illustrated in FIG. 4. Here, the importance Pᵢ (depicted as a solid line in FIG. 5) and the integrated value (depicted as a dashed line in FIG. 5) are written in each frame number of the in-vivo images included in the in-vivo image group. Further, in FIG. 5, only the range of the frame numbers “1” to “100” of the arranged in-vivo images having a large number of invalid areas is depicted and the other ranges are omitted. Further, in FIG. 5, the selected representative image is expressed as a black circle.

In the example illustrated in FIG. 5, the sum of the importance Pᵢ of sixty thousand in-vivo images is “10600.00” and the number of the representative images to be selected is “2000” similarly to the example illustrated in FIG. 4. For this reason, in step S5, “5.3” as a value obtained by dividing “10600.00” by “2000” is set as the first threshold value T similarly to the example illustrated in FIG. 4.

The integrated value at each time point of the frame numbers “1”, “23”, “47”, “67”, and “92” exceeds the integral multiple of the first threshold value T. For this reason, in step S8, the frame numbers “1”, “23”, “47”, “67”, and “92” are set as the boundary of the selection range. That is, in step S10, the in-vivo images of the frame numbers “1”, “23”, “47”, “67”, “92”, and the like are selected as the representative images.

As described above, in the first embodiment, the frame number as the integrated value exceeding the integral multiple of the first threshold value T is set as the boundary of the selection range and the in-vivo image of the frame number of the boundary is selected as the representative image.

Then, the representative images are selected at a substantially equal interval (the interval of the frame number) as illustrated in FIG. 4 in the arrangement range of the in-vivo images having a small number of invalid areas by the above-described selection. Even in the arrangement range of the in-vivo images having a large number of invalid areas, the representative images are selected at a substantially uniform interval as illustrated in FIG. 5 in the same way. Further, as understood by the comparison between FIGS. 4 and 5, the gap of the representative images selected in the arrangement range of the in-vivo images having a small number of invalid areas is narrower than the gap of the representative images selected in the arrangement range of the in-vivo images having a large number of invalid areas.

The image processing device 4 according to the above-described first embodiment sets a plurality of selection ranges based on the importance Pᵢ of each of the in-vivo images included in the in-vivo image group acquired in time sequence and selects the representative image every a plurality of selection ranges.

In particular, the image processing device 4 calculates a ratio of the number of the pixels as the valid area with respect to all pixels of the in-vivo image as the importance Pᵢ. Further, when the selection range needs to be set, the image processing device 4 integrates the importance Pᵢ of all in-vivo images in order of time sequence and sets the in-vivo image having the importance Pᵢ integrated at the last time when the integrated value exceeds the integral multiple of the first threshold value as the boundary of the selection range. Then, the image processing device 4 selects the in-vivo image as the boundary of the selection range as the representative image.

From the description above, the selection range in which the in-vivo images (the in-vivo images having a small number of invalid areas) having high importance Pᵢ are arranged can be narrower than the selection range in which the in-vivo images (the in-vivo images having a large number of invalid areas) having low importance Pᵢ are arranged (see FIGS. 4 and 5). That is, for example, when the number of the in-vivo images having high importance Pᵢ is equal to the number of the in-vivo images having low importance Pᵢ in the in-vivo image group, the number of the selection ranges is set to be comparatively large in the arrangement range of the in-vivo images having high importance Pᵢ and the number of the selection ranges set in the arrangement range of the in-vivo images having low importance Pᵢ can be decreased comparatively (see FIGS. 4 and 5).

Thus, there is an effect in which the in-vivo images having high importance Pᵢ can be mainly selected as the representative images from the in-vivo image group and the in-vivo images including many valid areas suitable for an observation can be selected as the representative images.

Further, the first threshold value is a value obtained by dividing the sum of the importance of all in-vivo images included in the in-vivo image group by the number of the representative images to be selected. For this reason, it is possible to select the representative images from all in-vivo images at a substantially uniform interval by a predicted number (see FIGS. 4 and 5).

Thus, a user can recognize the content of all in-vivo image groups by observing a predetermined number of the representative images subjected to the image summarization process.

### Second Embodiment

Next, a second embodiment of the invention will be described.

In the description below, the same reference numerals will be given to the same configurations and steps as those of the above-described first embodiment and the detailed description thereof will be omitted or simplified.

In the above-described first embodiment, the in-vivo image of the frame number as the boundary of the selection range is selected as the representative image.

On the contrary, in the second embodiment, the in-vivo image having the highest importance among the in-vivo images included in the selection range is selected as the representative image.

Then, the configuration of the image processing device according to the first embodiment is similar to the image processing device 4 described in the above-described first embodiment.

Hereinafter, only the image processing method according to the second embodiment will be described.

### Image Processing Method

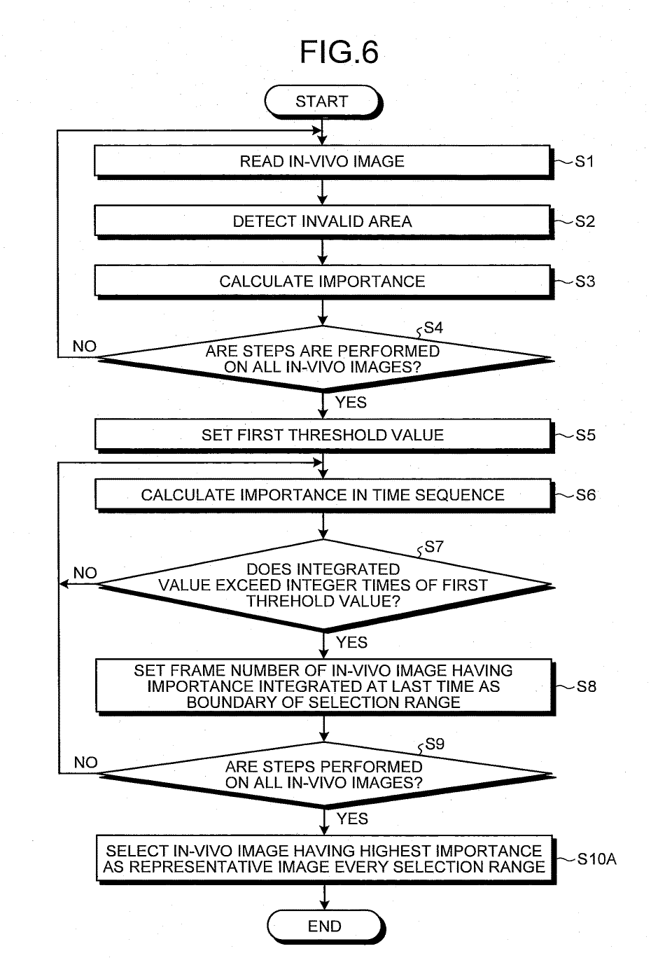

FIG. 6 is a flowchart illustrating the image processing method according to the second embodiment of the invention.

As illustrated in FIG. 6, the image processing method according to the second embodiment is different from the image processing method (FIG. 3) described in the above-described first embodiment in that step S10A is added instead of step S10. For this reason, only step S10A will be described below.

Step S10A is performed when it is determined that the above-described steps are performed on all in-vivo images in step S9 (step S9: Yes).

Specifically, in step S10A, the image selector 455 selects the in-vivo image having the highest importance among the in-vivo images included in the selection range every selection range having the boundary as the frame number (the frame number set as the boundary in step S8) stored in the memory unit 42 as the representative image.

### Detailed Example of Selected Representative Image

Next, a detailed example of the representative image selected by the image processing method according to the second embodiment will be described.

Hereinafter, in order to clarify a difference from the above-described first embodiment, a detailed example of the representative image selected by the image processing method according to the second embodiment will be described by using the in-vivo image group similar to the in-vivo image group exemplified in FIG. 4 and the in-vivo image group exemplified in FIG. 5.

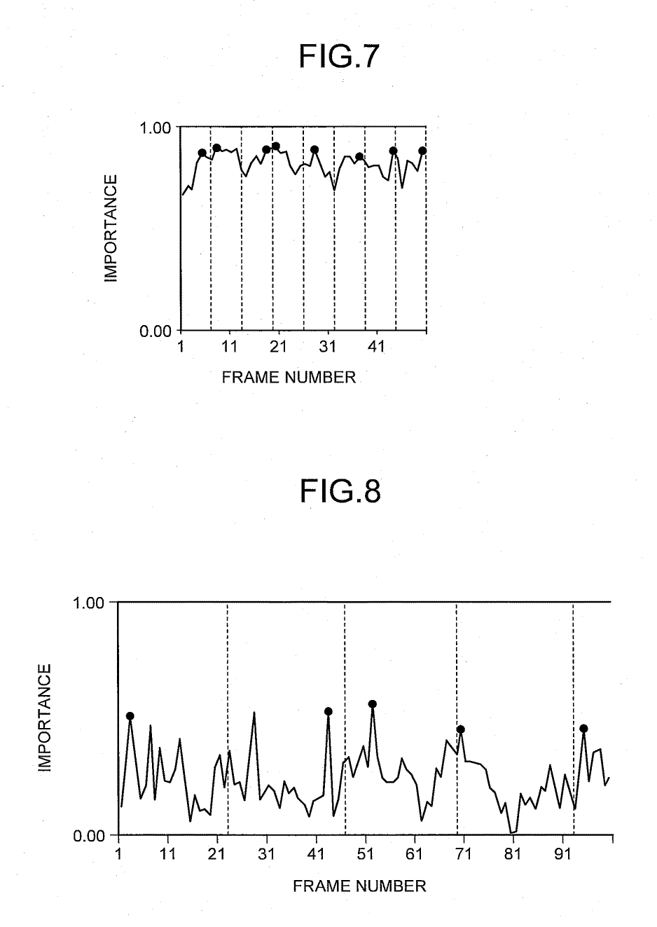

FIGS. 7 and 8 are diagrams illustrating an example of the representative image selected by the image processing method according to the second embodiment. Specifically, the in-vivo image group exemplified in FIG. 7 is the same as the in-vivo image group exemplified in FIG. 4. The in-vivo image group exemplified in FIG. 8 is the same as the in-vivo image group exemplified in FIG. 5. Furthermore, in FIGS. 7 and 8, the integrated value of the importance Pᵢ is not illustrated and only the importance Pᵢ is illustrated. Further, in FIGS. 7 and 8, the boundary of the selection range (in the example of FIG. 7, the frame numbers “7”, “13”, “19”, “26”, “32”, “38”, “and 45” and in the example of FIG. 8, the frame numbers “23”, “47”, “67”, and “92”) is depicted as a dashed line and the selected representative image is expressed as a black circle.

In the “arrangement range of the in-vivo images having a small number of invalid areas”, as illustrated in FIG. 7, in step S10A, the in-vivo images of the frame numbers “5”, “8”, “18”, “20”, “28”, “37”, “44”, “50”, and the like having the highest importance Pᵢ among the in-vivo images included in the selection range are respectively selected as the representative images every selection range.

Similarly, even in the “arrangement range of the in-vivo images having a large number of invalid areas”, as illustrated in FIG. 8, in step S10A, the in-vivo images of the frame numbers “3”, “43”, “52”, “70”, “95”, and the like having the highest importance Pᵢ among the in-vivo images included in the selection range are respectively selected as the representative images every selection range.

According to the above-described second embodiment, there are the following effects other than the effects of the above-described first embodiment.

The image processing device 4 according to the second embodiment selects the in-vivo image having the highest importance among the in-vivo images included in the selection range as the representative image.

Thus, in the above-described first embodiment, there is a concern that the in-vivo image having the smallest number of valid areas is selected as the representative image from the in-vivo images included in the selection range. However, according to the second embodiment, there is an effect that the in-vivo images having the largest number of valid areas can be selected as the representative images from the in-vivo images included in the selection range.

### Other Embodiments

So far, embodiments of the invention have been described, but the invention is not limited only by the above-described first and second embodiments.

In the above-described first and second embodiments, the image summarization process is performed on the in-vivo image group captured by the capsule endoscope 2, but the invention is not limited thereto. A configuration may be employed in which the image summarization process is performed on the other image groups as long as the image group is acquired in time sequence.

In the above-described first and second embodiments, the image processing device 4 acquires the in-vivo image group captured in time sequence in the capsule endoscope 2 by the recording medium 5 and the reader-writer 41, but the invention is not limited thereto.

For example, the in-vivo image group is stored in advance in a separately installed server. Further, the image processing device 4 is equipped with a communication unit that communicates with the server. Then, a configuration may be employed in which the image processing device 4 acquires the in-vivo image group by communicating with the server through the communication unit.

That is, the communication unit serves as an image acquiring unit that acquires the image data as the processing object from the outside.

In the above-described first and second embodiments, in step S6 to step S9, the importance Pᵢ of all in-vivo images are integrated in order of time sequence and the integrated value is compared with the integral multiple of the first threshold value, but the invention is not limited thereto.

For example, when the integrated value exceeds the first threshold value, the frame number of the in-vivo image having the importance Pᵢ integrated at the last time at the exceeding case is set as the boundary of the selection range. Further, the integrated value is initialized at the setting step. Then, the importance Pᵢ is integrated in order of time sequence again and the integrated value is compared with the first threshold value. Hereinafter, the above-described processes are repeated.

In the above-described first and second embodiments, the calculated importance may be adjusted based on the position of the pixel detected as the invalid area in the area detector 451 inside the in-vivo image.

For example, when the pixel detected as the invalid area is located at an area including the center inside the in-vivo image, the calculated importance is adjusted (changed) to a low value since there is an influence on the observation. Meanwhile, when the pixel detected as the invalid area is located at an outer edge area deviated from the center inside the in-vivo image, the calculated importance is adjusted (changed) to a high value since there is no influence on the observation.

As described above, since the importance is adjusted based on the position of the pixel detected as the invalid area, it is possible to appropriately set the importance of the in-vivo image in consideration of the influence during the observation.

In the above-described first and second embodiments, when the feature data used for the detection of the invalid area is the maximum value of the second threshold value, there is a high possibility that an erroneous detection may occur. For this reason, the second threshold value may be appropriately adjusted so as to be reliably detected as the invalid area.

Further, the process flow is not limited to the process flow of the flowchart described in the above-described first and second embodiments, and may be changed to a non-contradiction range.

Furthermore, an algorithm of a process described by using a flowchart in the specification can be described as a program. Such a program may be recorded in a recording unit inside a computer or may be recorded in a computer-readable recording medium. The program may be recorded in the recording unit or the recording medium when the computer or the recording medium is shipped as a product or may be downloaded via a communication network.

According to the image processing device, the image processing method, and the computer-readable recording medium of the disclosure, there is an effect that more images including many valid areas useful for an observation can be selected as representative images.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image processing device comprising:
a processor comprising hardware, wherein the processor is configured to:
detect an area not suitable for an observation inside each image included in an image group in which images are acquired in time sequence;
calculate an importance of the image for each of the images included in the image group based on the area not suitable for the observation inside the image;
integrate the importance in order of time sequence; and
determine whether the integrated value exceeds a threshold value.

2. The image processing device according to claim 1,
wherein the processor is further configured to set the image having the importance integrated at the last time when the integrated value exceeds the threshold value as a boundary of dividing the image group into a plurality of selection ranges if the processor determines that the integrated value exceeds the threshold value.

3. The image processing device according to claim 2,
wherein the processor is further configured to select a representative image from the images included in each of the selection ranges.

4. The image processing device according to claim 1,
wherein the area not suitable for the observation is an area other than an area suitable for the observation.

5. The image processing device according to claim 4,
wherein the area not suitable for the observation is at least one of an area in which residue or bubbles are reflected, an area in which a deep part of a lumen is reflected, a halation area, and a noise area.

6. The image processing device according to claim 1,
wherein the processor is configured to:
detect the area not suitable for the observation by unit of pixel, and
calculate the importance based on the number of pixels of the area not suitable for the observation.

7. The image processing device according to claim 6,
wherein the importance is a value obtained from Equation (1) as follows:
$${P}_{i} = 1 - \frac{count ( Invalid area )}{count ( All in - vivo images )} \qquad ( 1 )$$
where Pᵢ is the importance, count(Invalid area) is the number of pixels of the area not suitable for the observation, and count(All in-vivo images) is the number of all pixels of the image.

8. The image processing device according to claim 6,
wherein the processor is configured to calculate a high importance as the number of the pixels of the area not suitable for the observation decreases.

9. The image processing device according to claim 6,
wherein the processor is configured to adjust the calculated importance based on a position of the pixel of the area not suitable for the observation inside the image.

10. The image processing device according to claim 3,
wherein the processor is configured to select the image served as the boundary of the selection range as the representative image.

11. The image processing device according to claim 3,
wherein the processor is configured to select the image having the highest importance among the images included in the selection range as the representative image.

12. The image processing device according to claim 3,
wherein the threshold value is a value obtained by dividing the sum of the importance of all images included in the image group by the number of the representative images to be selected.

13. An image processing method executed by an image processing device, comprising:
detecting an area not suitable for an observation inside each image included in an image group in which images are acquired in time sequence;
calculating an importance of the image for each of the images included in the image group based on the area not suitable for the observation inside the image;
integrating the importance in order of time sequence; and
determining whether the integrated value exceeds a threshold value.

14. A non-transitory computer-readable recording medium recording a program for causing an image processing device to execute the image processing method according to claim 13.
